(19) United States
(12) Patent Application Publication
(10) Pub. No.: US 2016/0106840 A1
(43) Pub. Date: Apr. 21, 2016

(54) AQUEOUS BUFFER-FREE BIVALIRUDIN COMPOSITIONS

(71) Applicant: Eagle Pharmaceuticals, Inc., Woodcliff Lake, NJ (US)

(72) Inventors: Feng-Jing Chen, Irvine, CA (US); Steven L. Krill, Midland Park, NJ (US)

(21) Appl. No.: 14/982,813

(22) Filed: Dec. 29, 2015

Related U.S. Application Data
(63) Continuation of application No. 14/711,359, filed on May 13, 2015.
(60) Provisional application No. 61/992,375, filed on May 13, 2014.

(51) Int. Cl. A61K 47/02; A61K 9/08; A61K 38/58

(52) U.S. Cl. CPC A61K 47/02; A61K 38/58; A61K 9/08

(57) ABSTRACT

The present invention is directed to aqueous bivalirudin compositions and methods of administration of such compositions. The aqueous bivalirudin compositions of the present invention have a pH of about 3 to about 5 and are substantially free of buffer. Further compositions of the present invention include bivalirudin compositions comprising at least one stabilizer having a pH of about 3 to about 5. Additional compositions of the present invention include bivalirudin compositions comprising at least one stabilizer substantially free of buffer. Compositions of the present invention also include bivalirudin compositions substantially free of buffer. The invention is also directed to methods of treatment comprising administering a bivalirudin composition of the present invention. The invention is further directed to the use of the bivalirudin compositions of the present invention as an anticoagulant in a patient in need thereof.

This application claims the benefit of U.S. Provisional Application No. 61/992,375 filed May 13, 2014 which is incorporated by reference herein in its entirety.

### BACKGROUND OF THE INVENTION

Anticoagulants are substances that prevent blood from clotting. They are commonly used during percutaneous coronary intervention (“PCr”) and other catherization techniques in order to reduce bleeding complications during surgery. One class of anticoagulants is direct thrombin inhibitors that disrupt the activity of thrombin, a serine protease involved in the coagulation cascade that initiates clotting when fibrinogen is converted to fibrin. Thrombin also activates Factor XIII into Factor XIIIa (the latter which links fibrin polymers covalently), Factors V and VIII (which promote thrombin generation), and platelets (which help propagate the thrombus).

Bivalirudin directly inhibits thrombin by specifically binding to both its catalytic site and anion-binding exosite and is regarded as a highly effective anticoagulant for use during catherization procedures. Bivalirudin, also known as hirulog, is a synthetic congener of the naturally occurring thrombin peptide inhibitor hirudin, which is found in the saliva of the medicinal leech Hirudo medicinalis. Hirudin consists of 65 amino acids, although shorter peptide segments have proven to be effective as thrombin inhibitors. U.S. Pat. No. 5,196,404 (“'404 patent”, incorporated herein by reference) discloses bivalirudin among these shorter peptides that demonstrate anticoagulant activity. However, in contrast to hirudin, bivalirudin is a reversible thrombin inhibitor that is ideal for temporary prevention of blood clotting during catherization procedures.

Bivalirudin is a synthetic 20 amino acid peptide having the chemical name of D-Phenylalanyl-L-Prolyl-L-Arginyl-L-Prolyl-Glycyl-Glycyl-Glycyl-Glycyl-L-Asparagyl-Glycyl-L-Aspartyl-L-Phenylalanyl-L-Glutamyl-L- Glutamyl-L-Isoleucyl-L-Prolyl-L-Glutamyl-L-Glutamyl-L-Tyrosyl-L-Leucine trifluoroacetate (salt) hydrate and has a molecular weight of 2180 daltons (as the free base form). Bivalirudin is made up of the amino acid sequence: (D-Phe)-Pro-Arg-Pro-Gly-Gly-Gly-Gly-Asn-Gly-Asp-Phe-Glu-Glu-Ile-Pro-Glu-Glu-Tyr-Leu (SEQ ID NO: 1). As used herein, the term bivalirudin refers to the peptide comprising SEQ ID NO: 1, and salts thereof.

Peptides containing glutamine (“Gln”) and asparagine (“Asn”) residues, such as bivalirudin, in general, are not stable in aqueous solution and are not suitable for an RTU product due to the many mechanisms that result in degradation of the peptide in solution. These peptides commonly undergo a type of degradation reaction known as deamidation at susceptible Gln and, especially, Asn residues. Deamidation can be catalyzed at neutral and alkaline pH via a succinimide intermediate, but can also occur at acidic pH via direct hydrolysis of the side-chain amide group of the Asn residue. See Lindner et al., Exper. Geront. 36 (2001) 1551-1563 (“Linder et al.”). Consequently, it is difficult to prevent deamidation by simple pH adjustment. See generally WO 2009086062 at 1. Degradation via deamidation is one of the reasons why most peptide-based pharmaceuticals must be produced in lyophilized (i.e., freeze-dried) form, requiring reconstitution before injection or administration. See id. at 2.

The Asn residues of peptides and proteins, in neutral or alkaline solutions, can be deamidated via a cyclic succinimide (“cycloimido”) intermediate with release of ammonia (“NH₃”). See Lindner et al., at 1552. The cycloimido intermediate, if it can be detected at all, is usually present in only a very small amount. See id. at 1554. Hydrolysis of this intermediate can provide the corresponding peptides containing L-aspartic acid (“L-Asp”) and L-isoaspartic acid (“L-isoAsp”). See id at 1552. Moreover, racemization of the L-Asp-cycloimido peptide can lead to formation of the corresponding D-enantiomer, and after hydrolysis can lead to peptides containing D-Asp and D-isoAsp. See id. at 1553. Bischoff et al. describes the deamidation of recombinant hirudin in neutral and alkaline environments and suggests that hirudin undergoes the same deamidation mechanism. See Bischoff et al., Biochemistry 32 (1993) 725-734.

In acidic solutions, the mechanism of deamidation can be unpredictable since some peptides can undergo deamidation by direct hydrolysis (see id.) while other peptides can undergo deamidation by cycloimido intermediates (see Hekman et al., J. Pharm. Biomed. Anal., 20 (1999) 763-772, and references therein).

Notably, some cycloimido intermediates appear to be unstable (see Lindner et al.) while others appear to be stable (see Hekman et al.); as a result, the stability of cycloimido intermediates can be unpredictable.

Bivalirudin undergoes degradation by pathways that are common to other peptides, including D-Phe-Pro cleavage (resulting in [3-20]-bivalirudin), deamidation, hydrolysis, and isomerization. Bivalirudin is susceptible to deamidation via the Asn⁹-Gly¹⁰ residues that can form a [9-10]-cycloimido bivalirudin intermediate that can then undergo hydrolysis to Asp⁹-bivalirudin, isoAsp⁹-bivalirudin and isomers thereof. Asp⁹-bivalirudin is a known degradation and process impurity. See U.S. Patent Publication No. 20070093423 (“'423 publication”); U.S. application Ser. Nos. 12/180,550, 12/180,551, and 12/180,553. Controlling the formation of Asp⁹-bivalirudin has been an ongoing challenge, although recent improvements in the preparation of ANGIOMAX® have consistently controlled the formation of Asp⁹-bivalirudin generated during the compounding process. See U.S. application Ser. Nos. 12/180,550; 12/180,551; and 12/180,553.

ANGIOMAX® is a lyophilized powder that requires reconstitution prior to administration and has a pH of 5-6 (see ANGIOMAX® Prescribing Information). Once reconstituted, ANGIOMAX® may be stored at 2-8° C. for up to 24 hours. Diluted ANGIOMAX® may have a concentration of between 0.5 mg/mL and 5 mg/mL is stable at room temperature for up to 24 hours. See id. Thus, reconstituted, or diluted, ANGIOMAX® is not well suited for a ready to use product requiring long term storage stability. Furthermore, reconstitution can lead to dosing errors, wasted quantities of drug, inefficiencies in staff time, and constrained work flow.

Bivalirudin preparations have been described indicating that a pH above 5 is necessary for suitable chemical stability (U.S. Pat. No. 7,582,727 B1 and U.S. Pat. No. 7,598,343 B1). While Palepu et al., suggest that the pH of a bivalirudin solution between 4-5 may help stabilize the drug, pH alone was not adequate and the addition of a buffering agent is necessary to maintain stability (Palepu et al., U.S. Pat. No. 7,713,928 B1 and U.S. Pat. No. 7,803,762 B1).

Therefore, there is a need for stable, ready to use bivalirudin composition.

### SUMMARY OF THE INVENTION

In certain embodiments, the present invention is directed to aqueous bivalirudin compositions comprising bivalirudin and substantially free of buffer. Without being bound by a particular theory, bivalirudin may provide a self-buffering effect to the compositions of the invention. As used herein, “buffer” does not refer to any self-buffering effect provided by bivalirudin

In other embodiments, the present invention is directed to ready-to-use, aqueous bivalirudin compositions comprising bivalirudin and substantially free of buffer. The composition of the present invention can be further diluted in an appropriate diluent, such as, but not limited to, WFI (Water for Injection), 0.9% Sodium Chloride for Injection, or 5% Dextrose in Water, to a lower bivalirudin concentration for administration.

In certain embodiments, the present invention is directed to aqueous bivalirudin compositions comprising bivalirudin and one or more stabilizers. In particular embodiments, the stabilizer is present in an amount up to about 27 mg/mL, about 18 mg/mL, or about 9 mg/mL. In further embodiments, the stabilizer is present in an amount between about 8 mg/mL to about 16 mg/mL. In yet further embodiments, the stabilizer is present in at least about 10 mg/mL. In still further embodiments, the stabilizer is present in an amount such that the osmolality of the composition is about 200 to about 1000 mOsm/kg. In further embodiments, such compositions are ready-to-use.

In particular embodiments, the present invention is directed to aqueous bivalirudin compositions having a pH of about 3 to about 5 comprising bivalirudin.

In particular embodiments, the present invention is directed to ready-to-use, aqueous bivalirudin compositions having a pH of about 3 to about 5 comprising bivalirudin.

In other embodiments, the present invention is directed to aqueous bivalirudin compositions comprising bivalirudin and substantially free of buffer and having a pH of about 3 to about 5.

In other embodiments, the present invention is directed to aqueous bivalirudin compositions comprising bivalirudin and substantially free of buffer and having a pH of about 3 to about 5 In further embodiments, the invention is directed to aqueous bivalirudin compositions having a pH of about 3 to about 5 comprising bivalirudin and one or more stabilizers. In yet further embodiments, the stabilizer is present in an amount between about 8.0 mg/mL to about 16.0 mg/mL. In yet further embodiments, the stabilizer is present in at least about 27 mg/mL. In still further embodiments, the stabilizer is present in an amount such that the osmolality of the composition is about 200 to about 1000 mOsm/kg. In further embodiments, such compositions are ready-to-use.

In particular embodiments of the invention, aqueous bivalirudin compositions comprise bivalirudin and one or more stabilizers and are substantially free of buffer. In yet further embodiments, the stabilizer is present in an amount between about 8.0 mg/mL to about 16.0 mg/mL. In yet further embodiments, the stabilizer is present in at least about 27 mg/mL. In still further embodiments, the stabilizer is present in amount such that the osmolality of the composition is about 200 to about 1000 mOsm/kg. In further embodiments, such compositions are ready-to-use.

In certain embodiments, the present invention is directed to aqueous bivalirudin compositions having a pH of about 3 to about 5, comprising bivalirudin, and substantially free of buffer.

In certain embodiments, the present invention is directed to ready-to-use, aqueous bivalirudin compositions having a pH of about 3 to about 5, comprising bivalirudin, and substantially free of buffer.

In particular embodiments, the present invention is directed to aqueous bivalirudin compositions having a pH of about 3 to about 5, comprising bivalirudin, one or more stabilizers, and substantially free of buffer.

In particular embodiments, the present invention is directed to ready-to-use, aqueous bivalirudin compositions having a pH of about 3 to about 5, comprising bivalirudin, one or more stabilizers, and substantially free of buffer.

In further embodiments, the present invention is directed to aqueous bivalirudin compositions having a pH of about 3.9 to about 4.4, comprising bivalirudin, and substantially free of buffer.

In further embodiments, the present invention is directed to ready-to-use, aqueous bivalirudin compositions having a pH of about 3.9 to about 4.4, comprising bivalirudin, and substantially free of buffer.

In still further embodiments, the present invention is directed to aqueous bivalirudin compositions having a pH of about 3.9 to about 4.4, comprising bivalirudin, one or more stabilizers, and substantially free of buffer.

In still further embodiments, the present invention is directed to ready-to-use, aqueous bivalirudin compositions having a pH of about 3.9 to about 4.4, comprising bivalirudin, one or more stabilizers, and substantially free of buffer.

In yet further embodiments, the present invention is directed to aqueous bivalirudin compositions having a pH of about 4.2 to about 4.3, comprising bivalirudin, and substantially free of buffer.

In yet further embodiments, the present invention is directed to ready-to-use, aqueous bivalirudin compositions having a pH of about 4.2 to about 4.3, comprising bivalirudin, and substantially free of buffer.

In other embodiments, the present invention is directed to aqueous bivalirudin compositions having a pH of about 4.2 to about 4.3, comprising bivalirudin, one or more stabilizers, and substantially free of buffer.

In other embodiments, the present invention is directed to ready-to-use, aqueous bivalirudin compositions having a pH of about 4.2 to about 4.3, comprising bivalirudin, one or more stabilizers, and substantially free of buffer.

In still other embodiments, the present invention is directed to an aqueous bivalirudin composition having a pH of about 4.2 to about 4.3 comprising bivalirudin, 13 mg/mL of sodium chloride that is substantially free of buffer.

In still other embodiments, the present invention is directed to a ready-to-use, aqueous bivalirudin composition having a pH of about 4.2 to about 4.3 comprising bivalirudin, 13 mg/mL of sodium chloride that is substantially free of buffer.

Additional embodiments of the invention:
1. An aqueous bivalirudin composition comprising:

a) bivalirudin or salts thereof;

b) at least one or more stabilizers

wherein the composition has a pH of about 3 to about 5.
2. The composition of embodiment 1 comprising at least about 9 mg/mL of one or more stabilizers.
3. The composition of embodiment 1 comprising at least about 13 mg/mL of one or more stabilizers.
4. The composition of embodiment 1 wherein the stabilizer is an electrolyte, nonelectrolyte, or mixtures thereof.
5. The composition of embodiment 2 wherein the stabilizer is a nonelectrolyte selected from the group consisting of monosaccharides, disaccharides, sugar alcohols, and mixtures thereof.
6. The composition of embodiment 2 wherein the stabilizer is an electrolyte selected from the group consisting of uniunivalent electrolytes, diunivalent electrolytes, unidivalent electrolytes, and ditrivalent electrolytes, and mixtures thereof.
7. The composition of embodiment 6 wherein the stabilizer is sodium chloride.
8. The composition of embodiment 7 wherein sodium chloride is present in an amount from about 8 mg/mL to about 14 mg/mL.
9. The composition of embodiment 7 wherein sodium chloride is present in an amount from about 8 mg/mL to about 10 mg/mL.
10. The composition of embodiment 7 wherein sodium chloride is present in an amount from about 12 mg/mL to about 14 mg/mL.
11. The composition of embodiment 7 wherein sodium chloride is present in an amount from about 9 mg/mL.
12. The composition of embodiment 7 wherein sodium chloride is present in an amount from about 13 mg/mL.
13. The composition of embodiment 1 wherein the pH is about 3.9 to about 4.4.
14. The composition of embodiment 1 wherein the pH is about 4.2 to about 4.3
15. The composition of embodiment 1 wherein the pH is about 4.25.
16. The composition of embodiment 8 wherein the pH is about 3.9 to about 4.4.
17. The composition of embodiment 8 wherein the pH is about 4.2 to about 4.3
18. The composition of embodiment 8 wherein the pH is about 4.25.
19. The composition of embodiment 1 wherein the composition is substantially free of buffer.
20. The composition of embodiment 1 wherein the pH is about 3 to about 4.
21. The composition of embodiment 1 comprising about 1 mg/mL to about 10 mg/mL bivalirudin or a salt thereof.
22. The composition of embodiment 1 wherein the composition is stable.
23. The composition of embodiment 1 wherein the composition has a shelf-life of at least about 12 months.
24. The composition of embodiment 1 wherein no more than about 5% of bivalirudin is degraded upon storage at a temperature of about 2° C. to about 8° C. over 12 months.
25. An aqueous bivalirudin composition comprising:

a) bivalirudin or salts thereof and

b) at least one or more stabilizers,

wherein the composition is substantially free of buffer.
26. The composition of embodiment 25 wherein the stabilizer is an electrolyte, nonelectrolyte, or mixtures thereof.
27. The composition of embodiment 26 wherein the stabilizer is a nonelectrolyte selected from the group consisting of monosaccharides, disaccharides, sugar alcohols, and mixtures thereof.
28. The composition of embodiment 27 wherein the stabilizer is an electrolyte selected from the group consisting of uniunivalent electrolytes, diunivalent electrolytes, unidivalent electrolytes, and ditrivalent electrolytes, and mixtures thereof.
29. The composition of embodiment 27 wherein the stabilizer is sodium chloride.
30. The composition of embodiment 29 wherein sodium chloride is present in an amount from about 8 mg/mL to about 14 mg/mL.
31. The composition of embodiment 29 wherein sodium chloride is present in an amount from about 8 mg/mL to about 10 mg/mL.
32. The composition of embodiment 29 wherein sodium chloride is present in an amount from about 12 mg/mL to about 14 mg/mL.
33. The composition of embodiment 29 wherein sodium chloride is present in an amount from about 9 mg/mL.
34. The composition of embodiment 29 wherein sodium chloride is present in an amount from about 13 mg/mL.
35. The composition of embodiment 25 wherein the pH is about 3 to about 5.
36. The composition of embodiment 35 wherein the pH is about 3.9 to about 4.4.
37. The composition of embodiment 35 wherein the pH is about 4.25.
38. The composition of embodiment 35 wherein the pH is about 3 to about 4.
39. The composition of embodiment 25 wherein the composition is stable.
40. The composition of embodiment 25 wherein the composition has a shelf-life of at least about 12 months.
41. The composition of embodiment 25 wherein no more than about 5% of bivalirudin is degraded upon storage at a temperature of about 2° C. to about 8° C. over 12 months.
42. An aqueous bivalirudin composition comprising bivalirudin or salts thereof, wherein the composition is substantially free of buffer and has a pH of about 3 to about 5.
43. The composition of embodiment 42 wherein the pH is about 3.9 to about 4.4
44. The composition of embodiment 42 wherein the pH is about 4.25.
45. The composition of embodiment 42 further comprising at least one or more of a stabilizer.
46. The composition of embodiment 45 wherein the stabilizer is sodium chloride.
47. The composition of embodiment 46 comprising 6 mg/mL to 14 mg/mL of sodium chloride.
48. The composition of embodiment 47 wherein sodium chloride is present in an amount from about 8 mg/mL to about 10 mg/mL.
49. The composition of embodiment 47 wherein sodium chloride is present in an amount from about 12 mg/mL to about 14 mg/mL.
50. The composition of embodiment 47 wherein sodium chloride is present in an amount from about 9 mg/mL.
51. The composition of embodiment 47 wherein sodium chloride is present in an amount from about 13 mg/mL.
52. The composition of embodiment 42 further comprising a stabilizer in an amount such that the osmolality of the composition is about 200 to about 1000 mOsm/kg.
53. The composition of embodiment 42 wherein the composition is stable.
54. The composition of embodiment 42 wherein the composition has a shelf-life of at least about 12 months.
55. The composition of embodiment 42 wherein no more than about 5% of bivalirudin is degraded after storage at 2° C.-8° C. for about 12 months.
56. An aqueous bivalirudin composition comprising:

a) bivalirudin or salts thereof; and

b) one or more stabilizers an amount such that the osmolality of the composition is about 200 to about 1000 mOsm/kg

wherein the composition has a pH of about 3 to about 5.
57. The composition of embodiment 56 wherein the stabilizer is an electrolyte, nonelectrolyte, or mixtures thereof.
58. The composition of embodiment 57 wherein the stabilizer is a nonelectrolyte selected from the group consisting of monosaccharides, disaccharides, sugar alcohols, and mixtures thereof.
59. The composition of embodiment 57 wherein the stabilizer is an electrolyte selected from the group consisting of uniunivalent electrolytes, diunivalent electrolytes, unidivalent electrolytes, and ditrivalent electrolytes, and mixtures thereof.
60. The composition of embodiment 59 wherein the stabilizer is sodium chloride.
61. The composition of embodiment 60 wherein sodium chloride is present in an amount such that the osmolality of the composition is about 300 to about 500 mOsm/kg.
62. The composition of embodiment 56 wherein the pH is about 3.9 to about 4.4.
63. The composition of embodiment 56 wherein the pH is about 4.2 to about 4.3.
64. The composition of embodiment 56 wherein the pH is about 4.25.
65. The composition of embodiment 61 wherein the pH is about 3.9 to about 4.4.
66. The composition of embodiment 61 wherein the pH is about 4.2 to about 4.3.
67. The composition of embodiment 61 wherein the pH is about 4.25.
68. The composition of embodiment 56 wherein the composition is substantially free of buffer.
69. The composition of embodiment 56 wherein the composition is stable.
70. The composition of embodiment 56 wherein the composition has a shelf-life of at least about 12 months.
71. The composition of embodiment 56 wherein no more than about 5% of bivalirudin is degraded after storage at 2° C.-8° C. for about 12 months.
72. A stable aqueous bivalirudin composition comprising:

a) bivalirudin or salts thereof and

b) one or more stabilizers an amount such that the osmolality of the composition is about 200 to about 1000 mOsm/kg

wherein the composition is substantially free of buffer.
73. The composition of embodiment 72 wherein the stabilizer is an electrolyte, nonelectrolyte, or mixtures thereof.
74. The composition of embodiment 73 wherein the stabilizer is a nonelectrolyte selected from the group consisting of monosaccharides, disaccharides, sugar alcohols, and mixtures thereof.
75. The composition of embodiment 74 wherein the stabilizer is an electrolyte selected from the group consisting of uniunivalent electrolytes, diunivalent electrolytes, unidivalent electrolytes, and ditrivalent electrolytes, and mixtures thereof.
76. The composition of embodiment 75 wherein the stabilizer is sodium chloride.
77. The composition of embodiment 76 wherein sodium chloride is present in an amount such that the osmolality of the composition is about 300 to about 500 mOsm/kg.
78. The composition of embodiment 72 wherein the composition is stable.
79. The composition of embodiment 72 wherein the composition has a shelf-life of at least about 12 months.
80. The composition of embodiment 72 wherein no more than about 5% of bivalirudin is degraded after storage at 2° C.-8° C. for about 12 months.
81. An aqueous bivalirudin composition comprising

a) 5 mg/mL bivalirudin, and

b) 13 mg/mL sodium chloride,

wherein the composition is substantially free of buffer and has a pH of about 3.9 to about 4.4.
82. The composition of embodiment 81 wherein the composition is stable.
83. The composition of embodiment 81 wherein the composition has a shelf-life of at least about 12 months.
84. The composition of embodiment 81 wherein no more than about 5% of bivalirudin is degraded after storage at 2° C.-8° C. for about 12 months.
85. An aqueous bivalirudin composition comprising:

a) bivalirudin or salts thereof;

b) a carrier that is substantially free of buffer.
86. The composition of embodiment 85 wherein the composition has a pH of about 3 to about 5.
87. The composition of embodiment 85 wherein the composition further comprises a stabilizer that is selected from a group consisting of an electrolyte, nonelectrolyte, or mixtures thereof.
88. The composition of embodiment 87 wherein the stabilizer is a nonelectrolyte selected from the group consisting of monosaccharides, disaccharides, sugar alcohols, and mixtures thereof.
89. The composition of embodiment 87 wherein the stabilizer is an electrolyte selected from the group consisting of uniunivalent electrolytes, diunivalent electrolytes, unidivalent electrolytes, and ditrivalent electrolytes, and mixtures thereof.
90. The composition of embodiment 89 wherein the stabilizer is sodium chloride.
91. The composition of embodiment 90 wherein sodium chloride is present in an amount up to about 27 mg/mL.
92. The composition of embodiment 91 wherein sodium chloride is present in an amount of about 13 mg/mL.
93. The composition of embodiment 86 wherein the pH is about 4.
94. The composition of embodiment 86 wherein the pH is about 3.9 to about 4.4.
95. The composition of embodiment 86 wherein the pH is about 4.2 to about 4.3.
96. The composition of embodiment 86 wherein the pH is about 4.25.
97. The composition of embodiment 86 wherein the pH is about 3 to about 4.
98. An aqueous bivalirudin composition comprising:

a) bivalirudin or salts thereof;

b) at least one or more stabilizers,

wherein the composition has a pH of about 3 to about 5 and is substantially free of buffer.
99. The composition of embodiment 98 wherein the composition is stable.
100. The composition of embodiment 98 wherein the composition has a shelf-life of at least about 12 months.
101. A composition of embodiments 1-20, 22-80, or 85-100 comprising about 1 mg/mL to about 10 mg/mL bivalirudin or a salts thereof.
102. The composition of embodiment 101 comprising about 2.5 mg/mL to about 7.5 mg/mL.
103. The composition of embodiment 102 comprising about 5 mg/mL.
104. A composition of embodiments 1-20, 22-80, or 85-100 having a labeled bivalirudin concentration of 0.4-0.5 mg/mL.
105. A composition of embodiments 1-20, 22-80, or 85-100 having a labeled bivalirudin concentration of 1.0-10.0 mg/mL.
106. A composition of embodiment 105 wherein the labeled bivalirudin concentration is 5.0 mg/mL.
107. A composition of embodiment 105 wherein, the composition retains at least about 96%, or 950%, or 94%, or 93% or 92%, 91%, or 90.0% of the labeled bivalirudin concentration after storage at a temperature of about 2° C. to about 8° C. over 3, 6, 9, or 12 months.
108. A composition of embodiment 105 wherein the composition retains at least about 90% of the labeled bivalirudin concentration after storage at a temperature of about 2° C. to about 8° C. over 3, 6, 9, or 12 months.
109. A composition of embodiment 105 wherein the composition retains at least about 94% of the labeled bivalirudin concentration after storage at a temperature of about 2° C. to about 8° C. over 3, 6, 9, or 12 months.
110. A composition of embodiment 105 wherein the composition retains at least about 95% of the labeled bivalirudin concentration after storage at a temperature of about 2° C. to about 8° C. over 3, 6, 9, or 12 months.
111. A composition of embodiment 105 wherein the composition retains at least about 97% of the labeled bivalirudin concentration after storage at a temperature of about 2° C. to about 8° C. over 6 months.
112. A composition of embodiment 105 wherein the composition retains at least about 99% of the labeled bivalirudin concentration after storage at a temperature of about 2° C. to about 8° C. over 3 months.
113. A composition of embodiment 106 wherein, the composition retains at least about 96%, or 95%, or 94%, or 93% or 92%, 91%, or 90.0% of the labeled bivalirudin concentration after storage at a temperature of about 2° C. to about 8° C. over 3, 6, 9, or 12 months.
114. A composition of embodiment 106 wherein the composition retains at least about 90% of the labeled bivalirudin concentration after storage at a temperature of about 2° C. to about 8° C. over 3, 6, 9, or 12 months.
115. A composition of embodiment 106 wherein the composition retains at least about 94% of the labeled bivalirudin concentration after storage at a temperature of about 2° C. to about 8° C. over 3, 6, 9, or 12 months.
116. A composition of embodiment 106 wherein the composition retains at least about 95% of the labeled bivalirudin concentration after storage at a temperature of about 2° C. to about 8° C. over 3, 6, 9, or 12 months.
117. A composition of embodiment 106 wherein the composition retains at least about 97% of the labeled bivalirudin concentration after storage at a temperature of about 2° C. to about 8° C. over 6 months.
118. A composition of embodiment 106 wherein the composition retains at least about 99% of the labeled bivalirudin concentration after storage at a temperature of about 2° C. to about 8° C. over 3 months.
119. A composition of embodiment 104 wherein, the composition retains at least about 96%, or 95%, or 94%, or 93% or 92%, 91%, or 90.0% of the labeled bivalirudin concentration after storage at a temperature of about 2° C. to about 8° C. over 3, 6, 9, or 12 months.
120. A composition of embodiment 104 wherein the composition retains at least about 90% of the labeled bivalirudin concentration after storage at a temperature of about 2° C. to about 8° C. over 3, 6, 9, or 12 months.
121. A composition of embodiment 104 wherein the composition retains at least about 94% of the labeled bivalirudin concentration after storage at a temperature of about 2° C. to about 8° C. over 3, 6, 9, or 12 months.
122. A composition of embodiment 104 wherein the composition retains at least about 95% of the labeled bivalirudin concentration after storage at a temperature of about 2° C. to about 8° C. over 3, 6, 9, or 12 months.
123. A composition of embodiment 104 wherein the composition retains at least about 97% of the labeled bivalirudin concentration after storage at a temperature of about 2° C. to about 8° C. over 6 months.
124. A composition of embodiment 104 wherein the composition retains at least about 99% of the labeled bivalirudin concentration after storage at a temperature of about 2° C. to about 8° C. over 3 months.
125. A composition of any of embodiments 1-124 wherein the composition is a ready-to-use composition.
126. A method of treatment comprising administering a therapeutically effective amount of a composition of any of embodiments 1-125 to a patient in need thereof.
127. Use of a composition of any of embodiments 1-125 as an anticoagulant in a patient in need thereof.
128. A ready-to-use, aqueous bivalirudin composition comprising

a) 5 mg/mL bivalirudin, and

b) 13 mg/mL sodium chloride,

wherein the composition is substantially free of buffer and has a pH of about 4.2-4.3.
126. A method of treatment comprising administering a therapeutically effective amount of a ready-to-use, aqueous bivalirudin composition comprising

a) 5 mg/mL bivalirudin, and

b) 13 mg/mL sodium chloride,

wherein the composition is substantially free of buffer and has a pH of about 4.2-4.3.
127. Use of a ready-to-use, aqueous bivalirudin composition comprising

a) 5 mg/mL bivalirudin, and

b) 13 mg/mL sodium chloride,

wherein the composition is substantially free of buffer and has a pH of about 4.2-4.3 as an anticoagulant in a patient in need thereof.

### BRIEF DESCRIPTION OF THE FIGURES

FIGS. 1A & 1B depict the stability profile of bivalirudin as a function of pH.

FIG. 2 depicts the stability profile of bivalirudin as a function of pH and buffer at 60° C. for 48 hours.

FIG. 3 depicts the stability profile of bivalirudin as a function of pH and buffer upon storage at 40° C. for 7 days.

FIG. 4 depicts the stability profile of bivalirudin as a function of sodium chloride concentration at pH 4.25.

FIG. 5 depicts the stability profile of bivalirudin as a function of sodium chloride concentration at pH 4.50.

FIG. 6 depicts the effect of partially or completely replacing sodium chloride with sucrose, dextrose, or glycerin on the stability profile of bivalirudin.

FIG. 7 depicts the stability of bivalirudin at a given pH, i.e. pH 4.25 of variant F as illustrated by the figure can be improved by removing the buffer from the variant A. The stability of variant F can be further improved by higher sodium chloride concentration in variant L at the same pH.

### DETAILED DESCRIPTION

Unless defined otherwise, all technical and scientific terms used herein have the same meaning as is commonly understood by one of ordinary skill in the art to which this invention belongs. In the event that there is a plurality of definitions for a term used herein, those definitions in this section prevail unless stated otherwise.

### DEFINITIONS

As used herein “Area Under the Curve” or “AUC” refers to the area under an HPLC peak.

As used herein, the term “area percent” refers to the area percent of a particular HPLC peak relative to the total area of all relevant HPLC peaks. In certain embodiments, the area percent of a particular bivalirudin-related impurity is the percent area of said impurity's HPLC peak relative to the total peak area of bivalirudin and all bivalirudin-related impurities.

As used herein, the term “labeled bivalirudin concentration” refers to the bivalirudin concentration of the RTU dosage form. The terms “label claim” or “bivalirudin concentration label claim” can be used interchangeably with labeled bivalirudin concentration.

As used herein, a “ready-to-use” or “RTU” composition is a sterile, aqueous or non-aqueous or a combination thereof, injectable composition that is stable and has not been reconstituted from a lyophilizate within one day prior to use. In some embodiments, the composition of the present invention can be further diluted in an appropriate diluent, such as, but not limited to, WFI (Water for Injection), 0.9% Sodium Chloride for Injection, or 5% Dextrose in Water to a lower bivalirudin concentration for administration.

As used herein, a “sterile” composition is one in which essentially all forms of microbial life have been destroyed by an appreciable amount to meet the sterilization criteria outlined in the U.S. Pharmacopeia. See U.S. Pharmacopeia 32, NF 27, 1 (2009) 80-86.

As used herein, a “stable” composition does not exhibit appreciable degradation upon storage over a set time limit, at a set temperature, and at an identified pH or within an identified pH range. In certain embodiments, a stable composition means a composition retains at least about 90%, or at least about 91%, or at least about 92%, or at least about 93%, or at least about 94%, or at least about 95%, or at least about 96%, of the labeled bivalirudin concentration over 18 months, over 15 months, over 14 months, over 12 months, over 10 months, over 9 months, over 8 months, over 6 months, over 5 months, over 4 months, over 3 months, over 2 months, over 2 month, over 2 weeks, or over 1 week. In further embodiments, a stable composition retains a significant percentage of its labeled bivalirudin concentration upon storage at about 60° C., about 40° C., about 25° C., or about 2° C. to about 8° C. In certain embodiments, a stable composition does not exhibit appreciable degradation over 18 months, over 15 months, over 14 months, over 12 months, over 10 months, over 9 months, over 8 months, over 6 months, over 5 months, over 4 months, over 3 months, over 2 months, over 2 month, over 2 weeks, or over 1 week. In further embodiments, a stable composition does not exhibit appreciable degradation upon storage at about 60° C., about 40° C., about 25° C., or about 2° C. to about 8° C. In still further embodiments, appreciable degradation means at least about 15% (area percent), or at least about 12% (area percent), or at least about 10% (area percent), or at least about 9% (area percent), or at least about 7.5% (area percent), or at least about 6.5% (area percent), or at least about 6.0% (area percent) of bivalirudin-related impurities is found in the composition.

In still further embodiments, appreciable degradation means no more than about 15 wt % of bivalirudin is degraded, no more than about 12 wt % of bivalirudin is degraded, no more than about 10 wt % of bivalirudin is degraded, no more than about 8% of bivalirudin is degraded, or no more than about 5 wt % of bivalirudin is degraded. In yet further embodiments, appreciable degradation means a single impurity does not exceed about 15 AUC %, a single impurity does not exceed about 10 AUC %, a single impurity does not exceed about 5 AUC %, a single impurity does not exceed about 2.5 AUC %, or a single impurity does not exceed about 1 AUC %. In one embodiment, no more than about 15 wt % of bivalirudin is degraded upon storage at 25° C. over 1 month. In another embodiment, no more than about 12 wt % of bivalirudin is degraded upon storage at a temperature of about 2° C. to about 8° C. over 12 months. In another embodiment, no more than about 10 wt % of bivalirudin is degraded upon storage at a temperature of about 2° C. to about 8° C. over 12 months. In another embodiment, a single impurity does not exceed about 5 AUC % after storage at 25° C. for 1 month.

As used herein, the term “stabilizer” refers to any component that allows bivalirudin to be stable.

As used herein, the term “shelf-life” means a composition will retain approved specifications over a certain period of time. In particular embodiments, compositions have a shelf-life of about two years, about 18 months, about 12 months, or about six months.

As used herein, the term “has not been reconstituted from a lyophilizate” means that a solid has not been dissolved or suspended.

As used herein “substantially free” means less than about 0.05M. In particular embodiments, “substantially free” means less than about 0.045M, less than about 0.04M, less than about 0.035M, less than about 0.03M, less than about 0.025M, less than about 0.02 M, less than about 0.015 M, less than about 0.01M, less than about 0.008M, or less than about 0.005M.

As used herein, the term “bivalirudin-related substances” and “bivalirudin-related impurities” refer to cycloimido-bivalirudins and/or bivalirudin fragments other than bivalirudin and can be used interchangeably. Unless otherwise stated, as used herein, impurities refers to bivalirudin-related impurities

Bivalirudin

Bivalirudin can be synthesized by methods that include, but are not limited to, solid-phase peptide synthesis, solution-phase peptide synthesis, or a combination of solid-phase and solution-phase procedures (see, e.g., '404 patent; Okayama et al., Chem. Pharm. Bull., 44 (1996) 1344-1350; Steinmetzer et al., Eur. J. Biochem., 265 (1999) 598-605; '423 publication; U.S. Patent Publication No. 2008051558; U.S. Patent Publication No. 2008287648).

The bivalirudin in the compositions of the present invention may be the peptide encoded by SEQ ID NO: 1, or salts thereof. In certain embodiments of the invention, bivalirudin is present in an amount comprising between about 0.01 mg/mL and about 100 mg/mL, or between about 0.05 mg/mL and about 50 mg/mL, or between about 0.1 mg/mL and about 25 mg/mL, or between about 1.0 mg/mL and about 10 mg/mL, or between about 2.5 mg/mL and 7.5 mg/mL, such as a concentration of about 5.0 mg/mL. In certain embodiments of the invention, bivalirudin is present in an amount comprising between about 0.1 mg/mL and about 1.0 mg/mL, or between about 0.4 mg/mL and about 0.5 mg/mL, such as a concentration of about 0.45 mg/mL.

Cycloimido-Bivalirudin and Bivalirudin Fragments

In particular embodiments of the invention, the bivalirudin compositions may comprise one or more cycloimido-bivalirudins and/or bivalirudin fragments. In certain embodiments of the invention, the bivalirudin compositions may comprise [9-10]-cycloimido bivalirudin, [1]-12)-cycloimido bivalirudin, or a combination thereof. After storage at 25° C. for 1 month, [9-10]-cycloimido bivalirudin may be greater than 0.2% AUC to less than about 5% AUC, or may be greater than about 0.5% AUC to less than about 3% AUC, while the [11-12]-cycloimido bivalirudin may be greater than 0.1% AUC to less than about 5% AUC, or may be greater than about 1% AUC to less than about 4% AUC, or may be greater than about 2% AUC to less than about 3% AUC.

Similarly, after storage at 2-8° C. for 12 months, [9-10]-cycloimido bivalirudin may be greater than 0.2% AUC to less than about 5% AUC, or may be greater than about 0.5% AUC to less than about 3% AUC, and the [11-12]-cycloimido bivalirudin may be greater than 0.1% AUC to less than about 5% AUC, or may be greater than about 1% AUC to less than about 4% AUC, or may be greater than about 2% AUC to less than about 3% AUC.

In still other embodiments of the invention bivalirudin compositions may also comprise one or more bivalirudin fragments, i.e., bivalirudin-related impurities or bivalirudin-related substances, such as, but not limited to, L-Asp⁹-bivalirudin, [3-20]-bivalirudin, [1-11]-bivalirudin, [12-20]-bivalirudin, [5-20]-bivalirudin, [9-10]-cycloimido bivalirudin, [1]-12)-cycloimido bivalirudin, or combinations thereof.

Stabilizers

In certain embodiments of the invention, the compositions may comprise one or more pharmaceutically acceptable stabilizers that include, but are not limited to, nonelectrolytes, electrolytes, and mixtures thereof. Suitable nonelectrolytes include, but are not limited to, monosaccharides such as, but not limited to, glucose (dextrose), fructose, and galactose, disaccharides such as, but not limited to, sucrose, lactose, and maltose, and sugar alcohols, such as but not limited to, glycol, glycerol (glycerin), erythritol, propylene glycol, polyethylene glycol, and threitol. Suitable electrolytes include, but are not limited to, uniunivalent electrolytes, such as sodium chloride; diunivalent electrolytes, such as calcium chloride; unidivalent electrolytes, such as sodium sulfate; and ditrivalent electrolytes, such as sodium phosphate. In certain embodiments of the invention, the stabilizer is a salt formed between an acid and base that have a pKa of less than about 2.5 and greater than about 6.5, respectively. In another embodiment of the invention, the stabilizer is a sodium or calcium salt in which the conjugated acid has a pKa of less than about 2.5. In another embodiment of the invention, the stabilizer is a chloride salt in which the conjugated base has a pKa of greater than about 6.5.

In certain embodiments of the invention, at least one stabilizer is present in an amount up to about 27 mg/mL, up to about 18 mg/mL, up to about 13 mg/mL, up to about 9 mg/mL, up to about 6 mg/mL, up to about 3 mg/mL. In further embodiments of the invention, the stabilizer is present in an amount from about 3 mg/mL to about 16 mg/mL. In particular embodiments of the invention, the stabilizer is present in an amount from about 8 mg/mL to about 16 mg/mL. In further embodiments of the invention, the stabilizer is present in an amount between about 3 mg/mL to about 8 mg/mL, 8 mg/mL to about 10 mg/mL; about 8 mg/mL to about 12 mg/mL, about 8 mg/mL to about 14 mg/mL; about 10 mg/mL to about mg/mL to about 12 mg/mL; about 10 mg/mL to about 14 mg/mL; about 10 mg/mL to about 16 mg/mL; about 12 mg/mL to about 14 mg/mL, about 12 mg/mL to about 16 mg/mL; about 14 mg/mL to about 16 mg/mL; about 9 mg/mL to about 11 mg/mL, about 12 mg/mL to about 15 mg/mL.

In particular embodiments of the invention, at least one stabilizer is sodium chloride and is present in an amount up to about 27 mg/mL, up to about 18 mg/mL, up to about 13 mg/mL, up to about 9 mg/mL, up to 6 mg/mL, up to 3 mg/mL. In further embodiments of the invention, at least one stabilizer is sodium chloride and is present in 8 mg/mL to about 16 mg/mL. In still further embodiments of the invention, at least one stabilizer is sodium chloride and is present at about 13 mg/mL. In other embodiments of the invention, at least one stabilizer is sodium chloride and is present in at least about 9 mg/mL. In other embodiments of the invention, at least one stabilizer is sodium chloride and is present in at least about 6 mg/mL.

In particular embodiments of the invention, at least one stabilizer is sucrose and is present in an amount up to about 200 mg/mL, up to about 160 mg/mL, up to about 120 mg/mL or up to about 50 mg/mL, up to 30 mg/mL or up to 10 mg/mL. In particular embodiments of the invention, at least one stabilizer is dextrose and is present in an amount up to about 200 mg/mL, up to about 160 mg/mL, up to about 120 mg/mL, up to about 50 mg/mL, up to 30 mg/mL or up to 10 mg/mL. In particular embodiments of the invention, at least one stabilizer is glycerin and is present in an amount up to about 100 mg/mL, up to about 80 mg/mL, up to about 60 mg/mL or up to about 40 mg/mL, up to 20 mg/mL or up to 10 mg/mL.

In certain embodiments of the invention, the stabilizer is present in amount such that the osmolality of the composition is about 200 to about 1000 mOsm/kg. In particular embodiments of the invention, the stabilizer is present in amount such that the osmolality of the composition is about 200 to about 600 mOsm/kg; about 200 to about 500 mOsm/kg; about 200 to about 400 mOsm/kg; about 300 to about 600 mOsm/kg; about 300 to about 500 mOsm/kg; about 300 to about 400 mOsm/kg; about 400 to about 600 mOsm/kg; about 400 to about 500 mOsm/kg; or about 500 to about 600 mOsm/kg.

In certain embodiments of the invention, more than one stabilizers are present in amounts such that the osmolality of the composition is about 200 to about 1000 mOsm/kg. In particular embodiments of the invention, the stabilizer is present in amount such that the osmolality of the composition is about 200 to about 600 mOsm/kg; about 200 to about 500 mOsm/kg; about 200 to about 400 mOsm/kg; about 300 to about 600 mOsm/kg; about 300 to about 500 mOsm/kg; about 300 to about 400 mOsm/kg; about 400 to about 600 mOsm/kg; about 400 to about 500 mOsm/kg; or about 500 to about 600 mOsm/kg.

Buffer-Free

In particular embodiments of the invention, the composition is substantially free of buffer. Such buffers, include, but are not limited to ascorbate, lactobionate, gentisate, succinate, α-lipoic acid, maleate, chloroacetate, bicarbonate, tartrate, glycylglycine, formate, benzoate, citrate, lactate, acetate, phosphate, propionate, pyridine, piperazine, pyrophosphate, histidine, 2-(N-morpholino)ethanesulfonic acid (“MES”), cacodylic acid, (bis(2-hydroxyethyl)-imino-tris(hydroxymethyl)-methane) (“bis-TRIS”), bicarbonate, amino acids (glycine, glutamate, aspartate and etc) or a combination of these buffering agents. Without being bound by a particular theory, bivalirudin may provide a self-buffering effect to the compositions of the invention. As used herein, “buffer” does not refer to any self-buffering effect provided by bivalirudin.

In further embodiments, the compositions of the invention have less than about 0.05M, than about 0.045M, less than about 0.04M, less than about 0.035M, less than about 0.03M, less than about 0.025M, less than about 0.02 M, less than about 0.015 M, less than about 0.01M, less than about 0.008M, or less than about 0.005M of one or more buffers.

pH

In particular embodiments of the invention, the composition has a pH in the range of about 3 to about 5, including all subranges therebetween. In certain embodiments of the invention, the composition has a pH of about 3 to about 4.75; about 3 to about 4.5, about 3 to about 4.25; about 3 to about 4; about 3 to about 3.75; about 3 to about 3.5; about 3 to about 3.25; about 3.25 to about 5; about 3.25 to about 4.75; about 3.25 to about 4.5, about 3.25 to about 4.25; about 3.25 to about 4; about 3.25 to about 3.75; about 3.25 to about 3.5; about 3.5 to about 5; about 3.5 to about 4.75; about 3.5 to about 4.5, about 3.5 to about 4.25; about 3.5 to about 4; about 3.5 to about 3.75; about 3.75 to about 5; about 3.75 to about 4.75; about 3.75 to about 4.5, about 3.75 to about 4.25; about 3.75 to about 4; about 4 to about 5; about 4 to about 4.75; about 4 to about 4.5, about 4 to about 4.25; about 4.25 to about 5; about 4.25 to about 4.75; about 4.25 to about 4.5, about 4.5 to about 5; about 4.5 to about 4.75; or about 4.75 to about 5. In other embodiments of the invention, the composition has a pH of about 3.5; about 3.75; about 4; about 4.25; about 4.5; about 4.75, or about 5. In other embodiments of the invention, the composition has a pH of about 3 to about 4.4. In other embodiments of the invention, the composition has a pH of about 3.9 to about 4.4. In particular embodiments of the invention, the composition has a pH of about 4.2 to about 4.3.

In certain embodiments of the invention, the composition has a pH of about 3.5 to about 4.5 and comprises bivalirudin and one or more stabilizers in an amount between about 8 mg/mL to about 16 mg/mL. In further embodiments of the invention, the composition has a pH of about 3.5 to about 4.5 and comprises bivalirudin and one or more stabilizers in an amount between about 10 mg/mL to about 16 mg/mL. In still further embodiments of the invention, the composition has a pH of about 3.5 to about 4.5 and comprises bivalirudin and one or more stabilizers in an amount between about 12 mg/mL to about 16 mg/mL. In yet further embodiments of the invention, the composition has a pH of about 3.5 to about 4.5 and comprises bivalirudin and one or more stabilizers in an amount between about 13 mg/mL to about 15 mg/mL. In particular embodiments of the invention, the composition has a pH of about 3.5 to about 4.5 and comprises bivalirudin and one or more stabilizers in an amount of about 13 mg/mL.

In certain embodiments of the invention, the composition has a pH of about 3.75 to about 4.25 and comprises bivalirudin and one or more stabilizers in an amount between about 8 mg/mL to about 16 mg/mL. In further embodiments of the invention, the composition has a pH of about 3.75 to about 4.25 and comprises bivalirudin and one or more stabilizers in an amount between about 10 mg/mL to about 16 mg/mL. In still further embodiments of the invention, the composition has a pH of about 3.75 to about 4.25 and comprises bivalirudin and one or more stabilizers in an amount between about 12 mg/mL to about 16 mg/mL. In yet further embodiments of the invention, the composition has a pH of about 3.75 to about 4.25 and comprises bivalirudin and one or more stabilizers in an amount between about 13 mg/mL to about 15 mg/mL. In particular embodiments of the invention, the composition has a pH of about 3.75 to about 4.25 and comprises bivalirudin and one or more stabilizers in an amount of about 13 mg/mL.

In certain embodiments of the invention, the composition has a pH of about 3.5 to about 4.5 and comprises bivalirudin and one or more stabilizers in an amount such that the osmolality of the composition is about 200 to about 600 mOsm/kg. In further embodiments of the invention, the composition has a pH of about 3.5 to about 4.5 and comprises bivalirudin and one or more stabilizers in an amount such that the osmolality of the composition is about 300 to about 600 mOsm/kg. In still further embodiments of the invention, the composition has a pH of about 3.5 to about 4.5 and comprises bivalirudin and one or more stabilizers in an amount such that the osmolality of the composition is about 300 to about 500 mOsm/kg. In yet further embodiments of the invention, the composition has a pH of about 3.5 to about 4.5 and comprises bivalirudin and one or more stabilizers in an amount such that the osmolality of the composition is about 400 to about 600 mOsm/kg. In particular embodiments of the invention, the composition has a pH of about 3.5 to about 4.5 and comprises bivalirudin and one or more stabilizers in an amount such that the osmolality of the composition is about 400 to about 500 mOsm/kg.

In further embodiments of the invention, the composition has a pH of about 3 to about 4 and comprises bivalirudin and one or more stabilizers in an amount such that the osmolality of the composition is about 200 to about 600 mOsm/kg. In further embodiments of the invention, the composition has a pH of about 3.9 to about 4.4 and comprises bivalirudin and one or more stabilizers in an amount such that the osmolality of the composition is about 300 to about 500 mOsm/kg. In still further embodiments of the invention, the composition has a pH of about 3.75 to about 4.25 and comprises bivalirudin and one or more stabilizers in an amount such that the osmolality of the composition is about 300 to about 500 mOsm/kg. In yet further embodiments of the invention, the composition has a pH of about 3.9 to about 4.4 and comprises bivalirudin and one or more stabilizers in an amount such that the osmolality of the composition is about 200 to about 600 mOsm/kg.

In particular embodiments of the invention, the composition has a pH of about 3 to about 4 and comprises bivalirudin and sodium chloride and is substantially free of buffer. In certain embodiments of the invention, the composition has a pH of about 3 to about 4 and comprises about 5 mg/mL of bivalirudin and 9 mg/mL of sodium chloride and is substantially free of buffer.

In particular embodiments of the invention, the composition has a pH of about 3.9 to about 4.4 and comprises bivalirudin and sodium chloride and is substantially free of buffer. In certain embodiments of the invention, the composition has a pH of about 3.9 to about 4.4 and comprises about 5 mg/mL of bivalirudin and 9 mg/mL of sodium chloride and is substantially free of buffer.

In further embodiments of the invention, the composition has a pH of about 4.25 and comprises bivalirudin and sodium chloride and is substantially free of buffer. In still further embodiments of the invention, the composition has a pH of about 4.25 and comprises about 5 mg/mL of bivalirudin and 9 mg/mL of sodium chloride and is substantially free of buffer.

In particular embodiments of the invention, the composition has a pH of about 3 to about 4 and comprises bivalirudin and sodium chloride and is substantially free of buffer. In certain embodiments of the invention, the composition has a pH of about 3 to about 4 and comprises about 5 mg/mL of bivalirudin and 13 mg/mL of sodium chloride and is substantially free of buffer.

In particular embodiments of the invention, the composition has a pH of about 3.9 to about 4.4 and comprises bivalirudin and sodium chloride and is substantially free of buffer. In certain embodiments of the invention, the composition has a pH of about 3.9 to about 4.4 and comprises about 5 mg/mL of bivalirudin and 13 mg/mL of sodium chloride and is substantially free of buffer.

In further embodiments of the invention, the composition has a pH of about 4.25 and comprises bivalirudin and sodium chloride and is substantially free of buffer. In still further embodiments of the invention, the composition has a pH of about 4.25 and comprises about 5 mg/mL of bivalirudin and 13 mg/mL of sodium chloride and is substantially free of buffer.

In particular embodiments of the invention, the composition has a pH of about 3 to about 4 and comprises bivalirudin and sodium chloride and is substantially free of buffer. In certain embodiments of the invention, the composition has a pH of about 3 to about 4 and comprises about 5 mg/mL of bivalirudin, 6 mg/mL of sodium chloride and 50 mg/mL of dextrose and is substantially free of buffer.

In particular embodiments of the invention, the composition has a pH of about 3.9 to about 4.4 and comprises bivalirudin and sodium chloride and is substantially free of buffer. In certain embodiments of the invention, the composition has a pH of about 3.9 to about 4.4 and comprises about 5 mg/mL of bivalirudin, 6 mg/mL of sodium chloride and 50 mg/mL of dextrose and is substantially free of buffer.

In further embodiments of the invention, the composition has a pH of about 4.25 and comprises bivalirudin and sodium chloride and is substantially free of buffer. In still further embodiments of the invention, the composition has a pH of about 4.25 and comprises about 5 mg/mL of bivalirudin, 6 mg/mL of sodium chloride and 50 mg/mL of dextrose and is substantially free of buffer.

Stability and Shelf-Life

Certain embodiments of the invention are directed to stable bivalirudin compositions. In one embodiment, a stable composition retains appreciable percentage of the labeled bivalirudin concentration upon storage over a set time limit, at a set temperature, and at an identified pH or within an identified pH range. In certain embodiments, a stable composition retains at least about 90%, or at least about 91%, or at least about 92%, or at least about 93%, or at least about 94%, or at least about 95%, or at least about 96%, of the labeled bivalirudin concentration. In certain embodiments, a stable composition retains appreciable percentage of the labeled bivalirudin concentration over 18 months, over 15 months, over 14 months, over 12 months, over 10 months, over 9 months, over 8 months, over 6 months, over 5 months, over 4 months, over 3 months, over 2 months, over 1 month, over 2 weeks, or over 1 week. In further embodiments, a stable composition retains appreciable percentage of the labeled bivalirudin concentration upon storage at about 60° C., about 40° C., about 25° C., or about 2° C. to about 8° C. In certain embodiments, a stable composition does not exhibit appreciable degradation over 18 months, over 15 months, over 14 months, over 12 months, over 10 months, over 9 months, over 8 months, over 6 months, over 5 months, over 4 months, over 3 months, over 2 months, over 1 month, over 2 weeks, or over 1 week. In one embodiment, a stable composition does not exhibit appreciable degradation upon storage over a set time limit, at a set temperature, and at an identified pH or within an identified pH range. In further embodiments, a stable composition does not exhibit appreciable degradation upon storage at about 60° C., about 40° C., about 25° C., or about 2° C. to about 8° C. In still further embodiments, appreciable degradation means at least about 15%/o, or at least about 12%, or at least about 10%, or at least about 9%, or at least about 7.5%, or at least about 6.5%, or at least about 6.0% (area percent) of bivalirudin impurities is found in the composition.

In certain embodiments of the invention, the bivalirudin the labeled bivalirudin concentration is present in an amount comprising between about 0.01 mg/mL and about 100 mg/mL, or between about 0.05 mg/mL and about 50 mg/mL, or between about 0.1 mg/mL and about 25 mg/mL, or between about 1.0 mg/mL and about 10 mg/mL, or between about 2.5 mg/mL and 7.5 mg/mL, such as a concentration of about 5.0 mg/mL. In certain embodiments of the invention, the labeled bivalirudin concentration is present in an amount comprising between about 0.1 mg/mL and about 1.0 mg/mL, or between about 0.4 mg/mL and about 0.5 mg/mL, such as a concentration of about 0.45 mg/mL.

Certain embodiments of the invention are directed to stable bivalirudin compositions. In one embodiment, no more than about 15% of bivalirudin is degraded upon storage at 25° C. over 1 month. In another embodiment, no more than about 12%/o of bivalirudin is degraded upon storage at a temperature of about 2° C. to about 8° C. over 12 months. In yet another embodiment, no more than about 8% of bivalirudin is degraded upon storage at a temperature of about 2° C. to about 8° C. over 12 months. In still another embodiment, no more than about 7% of bivalirudin is degraded upon storage at a temperature of about 2° C. to about 8° C. over 12 months. In still another embodiment, no more than about 6% of bivalirudin is degraded upon storage at a temperature of about 2° C. to about 8° C. over 12 months. In still another embodiment, no more than about 5% of bivalirudin is degraded upon storage at a temperature of about 2° C. to about 8° C. over 12 months. In another embodiment, a single impurity does not exceed about 5% after storage at 25° C. for 1 month. In one embodiment, no more than about 10% of bivalirudin is degraded upon storage at 40° C. over 1 week.

In still further embodiments of the invention, the composition retains at least about 90% of the labeled bivalirudin concentration after storage at a temperature of about 2° C. to about 8° C. over 3 months. In yet further embodiments of the invention, the composition retains at least about 91% of the labeled bivalirudin concentration after storage at a temperature of about 2° C. to about 8° C. over 3 months. In particular embodiments of the invention, the composition retains at least about 92% of the labeled bivalirudin concentration after storage at a temperature of about 2° C. to about 8° C. over 3 months. In certain embodiments of the invention, the composition retains at least about 93% of the labeled bivalirudin concentration after storage at a temperature of about 2° C. to about 8° C. over 3 months. In still further embodiments of the invention, the composition retains at least about 94% of the labeled bivalirudin concentration after storage at a temperature of about 2° C. to about 8° C. over 3 months. In yet further embodiments of the invention, the composition retains at least about 95% of the labeled bivalirudin concentration after storage at a temperature of about 2° C. to about 8° C. over 3 months. In particular embodiments of the invention, the composition retains at least about 96% of the labeled bivalirudin concentration after storage at a temperature of about 2° C. to about 8° C. over 3 months. In certain embodiments of the invention, the composition retains at least about 97% of the labeled bivalirudin concentration after storage at a temperature of about 2° C. to about 8° C. over 3 months. In still further embodiments of the invention, the composition retains at least about 98% of the labeled bivalirudin concentration after storage at a temperature of about 2° C. to about 8° C. over 3 months. In yet further embodiments of the invention, the composition retains at least about 99% of the labeled bivalirudin concentration after storage at a temperature of about 2° C. to about 8° C. over 3 months. In particular embodiments of the invention, the composition retains at least about 99.5% of the labeled bivalirudin concentration after storage at a temperature of about 2° C. to about 8° C. over 3 months. In certain embodiments of the invention, the composition retains at least about 99.9% of the labeled bivalirudin concentration after storage at a temperature of about 2° C. to about 8° C. over 3 months.

In still further embodiments of the invention, the composition retains at least about 9.0% of the labeled bivalirudin concentration after storage at a temperature of about 2° C. to about 8° C. over 6 months. In yet further embodiments of the invention, the composition retains at least about 91% of the labeled bivalirudin concentration after storage at a temperature of about 2° C. to about 8° C. over 6 months. In particular embodiments of the invention, the composition retains at least about 92% of the labeled bivalirudin concentration after storage at a temperature of about 2° C. to about 8° C. over 6 months. In certain embodiments of the invention, the composition retains at least about 93% of the labeled bivalirudin concentration after storage at a temperature of about 2° C. to about 8° C. over 6 months. In still further embodiments of the invention, the composition retains at least about 94% of the labeled bivalirudin concentration after storage at a temperature of about 2° C. to about 8° C. over 6 months. In yet further embodiments of the invention, the composition retains at least about 95% of the labeled bivalirudin concentration after storage at a temperature of about 2° C. to about 8° C. over 6 months. In particular embodiments of the invention, the composition retains at least about 96% of the labeled bivalirudin concentration after storage at a temperature of about 2° C. to about 8° C. over 6 months. In certain embodiments of the invention, the composition retains at least about 97% of the labeled bivalirudin concentration after storage at a temperature of about 2° C. to about 8° C. over 6 months. In still further embodiments of the invention, the composition retains at least about 98% of the labeled bivalirudin concentration after storage at a temperature of about 2° C. to about 8° C. over 6 months. In yet further embodiments of the invention, the composition retains at least about 99% of the labeled bivalirudin concentration after storage at a temperature of about 2° C. to about 8° C. over 6 months. In particular embodiments of the invention, the composition retains at least about 99.5% of the labeled bivalirudin concentration after storage at a temperature of about 2° C. to about 8° C. over 6 months. In certain embodiments of the invention, the composition retains at least about 99.9% of the labeled bivalirudin concentration after storage at a temperature of about 2° C. to about 8° C. over 6 months.

In still further embodiments of the invention, the composition retains at least about 90% of the labeled bivalirudin concentration after storage at a temperature of about 2° C. to about 8° C. over 12 months. In yet further embodiments of the invention, the composition retains at least about 91% of the labeled bivalirudin concentration after storage at a temperature of about 2° C. to about 8° C. over 12 months. In particular embodiments of the invention, the composition retains at least about 92% of the labeled bivalirudin concentration after storage at a temperature of about 2° C. to about 8° C. over 12 months. In certain embodiments of the invention, the composition retains at least about 93% of the labeled bivalirudin concentration after storage at a temperature of about 2° C. to about 8° C. over 12 months. In still further embodiments of the invention, the composition retains at least about 94% of the labeled bivalirudin concentration after storage at a temperature of about 2° C. to about 8° C. over 12 months. In yet further embodiments of the invention, the composition retains at least about 95% of the labeled bivalirudin concentration after storage at a temperature of about 2° C. to about 8° C. over 12 months. In particular embodiments of the invention, the composition retains at least about 96% of the labeled bivalirudin concentration after storage at a temperature of about 2° C. to about 8° C. over 12 months. In certain embodiments of the invention, the composition retains at least about 97% of the labeled bivalirudin concentration after storage at a temperature of about 2° C. to about 8° C. over 12 months. In still further embodiments of the invention, the composition retains at least about 98% of the labeled bivalirudin concentration after storage at a temperature of about 2° C. to about 8° C. over 12 months. In yet further embodiments of the invention, the composition retains at least about 99% of the labeled bivalirudin concentration after storage at a temperature of about 2° C. to about 8° C. over 12 months. In particular embodiments of the invention, the composition retains at least about 99.5% of the labeled bivalirudin concentration after storage at a temperature of about 2° C. to about 8° C. over 12 months. In certain embodiments of the invention, the composition retains at least about 99.9% of the labeled bivalirudin concentration after storage at a temperature of about 2° C. to about 8° C. over 12 months.

Method of Treatment

In certain embodiments, the present invention is directed to a method of treatment comprising administering an aqueous bivalirudin composition described herein.

The bivalirudin composition can be an injectable dosage form, and can be delivered to the subject parenterally. Methods of delivering the bivalirudin compositions parenterally are well known in the art. For example, the aqueous composition may be delivered intravenously.

The aqueous composition may be administered in an intravenous bolus dose of between about 0.25 mg/kg and about 1.5 mg/kg, or between about 0.5 mg/kg to about 1 mg/kg, or about 0.75 mg/kg. This may be followed by an infusion of the aqueous composition of between about 1.25 mg/kg/h and about 2.25 mg/kg/h, or about 1.75 mg/kg/h for the duration of the procedure or treatment protocol. Five minutes after the bolus dose is administered, an additional bolus of between about 0.1 mg/kg and about 1 mg/kg, or about 0.3 mg/kg, may be given if needed.

The aqueous bivalirudin compositions of the present invention can be indicated for use as an anticoagulant. Also, the aqueous bivalirudin compositions of the present invention can be used for the prevention and treatment of venous thromboembolic disease. Likely indications include treatment in patients with unstable angina undergoing percutaneous transluminal coronary angioplasty; treatment in patients undergoing percutaneous coronary intervention (PCI) with provisional use of glycoprotein IIb/IIIa inhibitor (GPI) as in the REPLACE-2 study; administration with the provisional use of glycoprotein IIb/IIa inhibitor for use as an anticoagulant in patients undergoing PCI; and treatment in patients with, or at risk of, HIT or HITTS undergoing PCI. Also, aqueous bivalirudin compositions of the present invention can be used for the prevention and treatment of venous thromboembolic disease.

The aqueous bivalirudin compositions of the present invention may be administered with other drug products such as glycoprotein IIb/IIIa inhibitor (see, e.g., Allie et al., Iasc. Dis. Manage. 3 (2006) 368-375). Alternatively, aqueous bivalirudin compositions of the present invention may be combined with blood thinners including, but not limited to, coumadin, warfarin, and preferably, aspirin.

### EXAMPLES

### General Methods

Preparation of Bivalirudin Compositions

A series of laboratory-scale bivalirudin formulations were prepared as follows.

Formulations A and B were prepared by dissolving sodium acetate trihydrate and sodium chloride in water and adding bivalirudin slowly to this solution with stirring until completely dissolved. The pH was adjusted to a desired value using 2N acetic acid. The formulations were stored in 5 mL glass vials.

Formulations C, F, H, I, J, and L were prepared by dissolving sodium chloride in water and adding bivalirudin slowly or incrementally to this solution with stirring until completely dissolved. The pH was adjusted to a desired value using 2N acetic acid or 0.1 N NaOH. The formulations were stored in 5 mL glass vials.

Formulations D, E, M, and N were prepared by dissolving sucrose (formulations D and M) or dextrose (formulations E and N) in water and adding bivalirudin slowly to this solution with stirring until completely dissolved. The pH was adjusted to a desired value using 2N acetic acid or 0.1 N NaOH. The formulations were stored in 5 mL glass vials.

Formulation G was prepared by dissolving sodium chloride and dextrose in water and adding bivalirudin slowly to this solution with stirring. The pH was adjusted to a desired value using 0.1 N NaOH. The formulation was stored in 5 mL glass vials.

Formulation O was prepared by adding glycerin to water and adding bivalirudin slowly to this solution with stirring. The pH was adjusted to a desired value using 0.1 N NaOH. The formulation was stored in 5 mL glass vials.

Formulation K was prepared by adding bivalirudin in water with stirring. The pH was adjusted to a desired value using 0.1 N NaOH. The formulation was stored in 5 mL glass vials.

Formulations P, Q, and R were prepared by adjusting the pH of formulations L, J, and H respectively to a desired value using 0.1 N NaOH. The formulations were stored in 5 mL Type I clear glass vials, sealed by West 4432/50 Chlorobutyl stopper with Fluorotec coating and having air as headspace.

While the procedures described above typically have an order of addition by adding sodium chloride to water before the addition of bivalirudin, an alternative order of addition could be considered by adding bivalirudin in water first then either (1) adjusting pH to the desired value followed by the addition of sodium chloride or (2) adding sodium chloride next followed by pH adjustment to the desired value. Adding bivalirudin before sodium chloride has the potential advantage of decreasing the time required for API addition and/or API dissolution time therefore reducing the time of the batch exposed to the environment during compounding. Although a turbid drug solution might be observed in the latter upon addition of sodium chloride, adjusting the pH to the higher desired value by NaOH usually resulting in a clear drug solution effectively.

The presence of a non-ionic stabilizer such as dextrose unexpectedly improved the wetting and the dissolution of bivalirudin in water even in the presence of sodium chloride. Therefore, a shorter overall compounding time was observed with the formulation containing sucrose or dextrose such as Formulations D, E and G.

Stability Studies

Unless otherwise noted, samples of each formulation were prepared for the stability studies by filling 4 mL of the formulation in a 5 mL Type I clear glass vial sealed by West 4432/50 Chlorobutyl stopper with Fluorotec coating and having air as headspace. For stability measurements, unless otherwise noted, samples were stored at an upright position and tested for purity of bivalirudin, bivalirudin-related impurities, and pH. For selected formulations, the osmolality was also tested at time zero.

Osmolality Measurements

The instrument for measuring osmolality was standardized using purified water for 0 mOsm/kg, and using osmolality standards for 100 mOsm/kg, 500 mOsm/kg, and 1500 mOsm/Kg. The sample size for the analysis was 0.2 mL. Two replicate analyses of each formulation were performed and the mean value was reported for each formulation.

HPLC Method for Purity/Impurity Analysis of RTU Bivalirudin Compositions

The purity and impurities of bivalirudin compositions under various storage conditions were measured using HPLC. HPLC was performed using gradient of acetonitrile and water with sodium phosphate buffer at a pH of 6.5, a C18 column, a flow rate of 1.2 mL/min, a column temperature of 40° C., and a total runtime of 40 minutes including the post runtime. Absorbance at 215 nm was measured. Bivalirudin peak purity or impurities was reported as area percent.

### Example 1

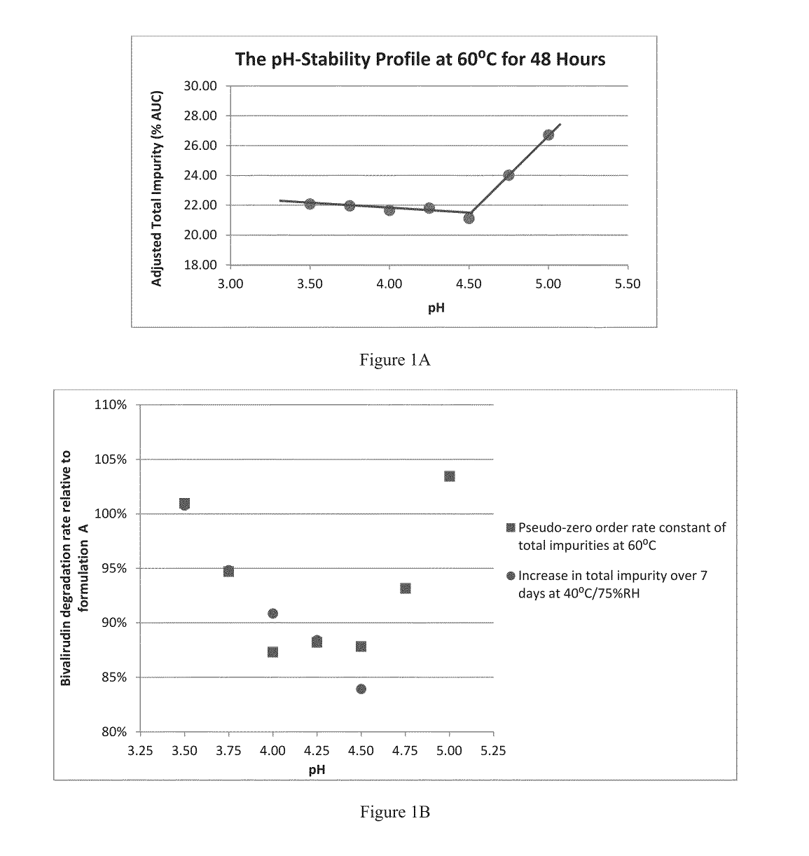

The stability profiles of bivalirudin in normal saline at 40° C. and 60° C. in a series of formulations with a pH range from 3.50 to 5.00 were measured. The formulations shown in Table 1 were prepared as described above. Impurities were measured as described above, and the results are shown in Table 2 (Total Impurity) and Table 3 (Adjusted Total Impurity). FIGS. 1A and 1B depict the stability profile of bivalirudin as a function of pH. In order to compare the stability profiles of the various formulations, the bivalirudin degradation rate of each formulation was normalized against that of Formulation A (Example 2). The pseudo-zero order rate constant at 60° C. based upon the accumulation of total impurity and the increase in total impurities over 7 days at 40° C./75% RH relative to those of Formulation A is shown in Table 4.

TABLE 1
Bivalirudin Formulations with Varying pH

| Composition | | | | | | | |
|---|---|---|---|---|---|---|---|
| (mg/mL) | J | C | H | F | I | Q | R |
| Bivalirudin* | 5.00 | 5.00 | 5.00 | 5.00 | 5.00 | 5.00 | 5.00 |
| Sodium | 9.00 | 9.00 | 9.00 | 9.00 | 9.00 | 9.00 | 9.00 |
| Chloride | | | | | | | |
| Hydrochloric | qs to | qs to | qs to | qs to | qs to | qs to | qs to |
| Acid | pH | pH | pH | pH | pH | pH | pH |
| | 3.50 | 3.75 | 4.00 | 4.25 | 4.50 | 4.75 | 5.00 |
| Sodium | qs to | qs to | qs to | qs to | qs to | qs to | qs to |
| Hydroxide | pH | pH | pH | pH | pH | pH | pH |
| | 3.50 | 3.75 | 400 | 4.25 | 4.50 | 4.75 | 5.00 |
| WFI | qs | qs | qs | qs | qs | qs | qs |

*Equivalent of anhydrous TFA-free form. The actual amount for dispensing should be calculated based upon the water and TFA content according to the Certificate of Analysis (COA).

TABLE 2
Total Impurities (% AUC) over 7 days
of Formulations J, C, H, F, I, Q & R

| | J | C | H | F | I (n = 2) | Q | R |
|---|---|---|---|---|---|---|---|
| Time | 0.53 | 0.65 | 0.57 | 0.54 | 0.63 | 0.85 | 0.99 |
| Zero | | | | | | | |
| 12 hr at | 6.22 | — | 6.65 | 6.41 | 6.97 | 8.63 | 9.58 |
| 60° C. | | | | | | | |
| 24 hr at | 11.70 | — | 12.26 | 12.04 | 12.39 | 14.28 | 15.89 |
| 60° C. | | | | | | | |
| 48 hr at | 22.62 | 22.61 | 22.22 | 22.34 | 21.75 | 24.86 | 27.71 |
| 60° C. | | | | | | | |
| 7 days at | 11.06 | 10.04 | 10.61 | 10.29 | 9.28 | — | — |
| 40° C. | | | | | | | |

TABLE 3
Adjusted Impurities (% AUC) over 7 days
of Formulations J, C, H, F, I, Q & R

| | J | C | H | F | I (n = 2) | Q | R |
|---|---|---|---|---|---|---|---|
| Time | 0.00 | 0.00 | 0.00 | 0.00 | 0.00 | 0.00 | 0.00 |
| Zero | | | | | | | |
| 12 hr at | 5.68 | — | 6.08 | 5.86 | 6.34 | 7.77 | 8.59 |
| 60° C. | | | | | | | |
| 24 hr at | 11.17 | — | 11.69 | 11.50 | 11.76 | 13.43 | 14.90 |
| 60° C. | | | | | | | |
| 48 hr at | 22.08 | 21.96 | 21.65 | 21.80 | 21.12 | 24.01 | 26.72 |
| 60° C. | | | | | | | |
| 7 days at | 10.52 | 9.39 | 10.04 | 9.75 | 8.78 | — | — |
| 40° C. | | | | | | | |

TABLE 4
Relative Bivalirudin Stability over 7 days of Formulations
J, C, H, F, I, Q & R as compared to Formulation A

| Composition (mg/mL) | J | C | H | F | I | Q | R |
|---|---|---|---|---|---|---|---|
| pH | 3.50 | 3.75 | 4.00 | 4.25 | 4.50 | 4.75 | 5.00 |
| Pseudo-zero order rate | 101% | 95% | 87% | 88% | 88% | 93% | 103% |
| constant of total | | | | | | | |
| impurities at 60° C. | | | | | | | |
| Increase in total | 101% | 95% | 91% | 88% | 84% | — | — |
| impurity over 7 days | | | | | | | |
| at 40° C./75% RH | | | | | | | |

### Example 2

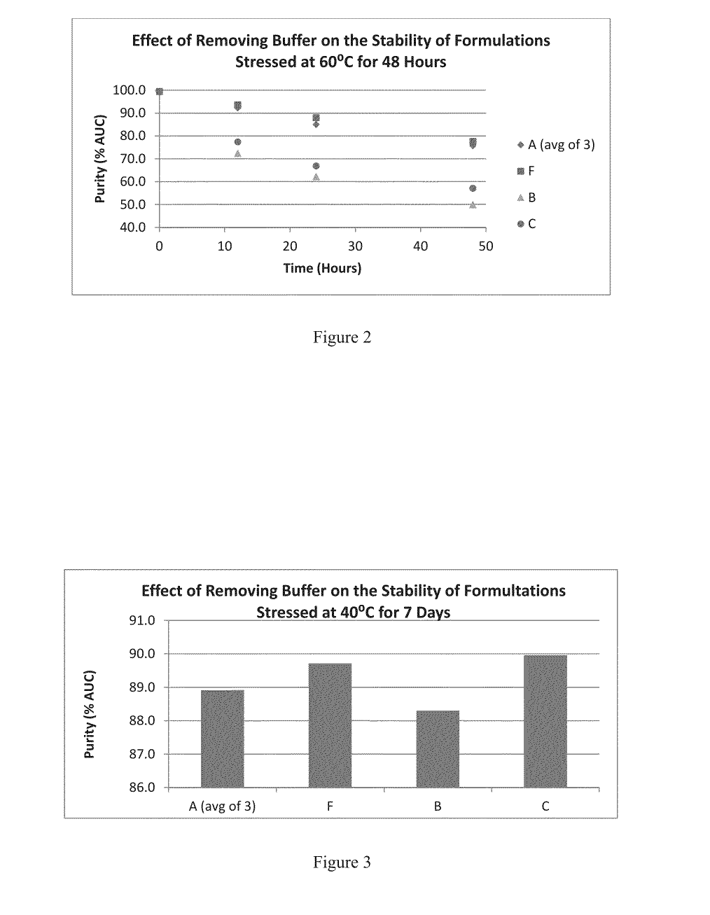

The stability profiles of a bivalirudin solution at two different pH levels in the presence and absence of a buffer were measured. The formulations shown in Table 5 were prepared as described above. Purity was measured as described above. The results are shown in Table 6; the relative stability (degradation rate) normalized against Formulation A are shown in Table 7. FIG. 2 depicts the effect of buffer on bivalirudin purity at pH 3.75 and 4.25 at 60° C. for 48 hours. FIG. 3 depicts the effect of buffer on bivalirudin purity at pH 3.75 and 4.25 at at 40° C. for 7 days.

TABLE 5
Bivalirudin Formulation Composition

| Composition (mg/mL) | A | F | B | C |
|---|---|---|---|---|
| Bivalirudin* | 5.00 | 5.00 | 5.00 | 5.00 |
| Sodium Acetate Trihydrate | 6.48 | — | 6.48 | — |
| Sodium Chloride | 9.00 | 9.00 | 9.00 | 9.00 |
| Glacial Acetic Acid | qs to pH 4.25 | — | qs to pH 3.75 | — |
| Hydrochloric Acid | — | qs to pH 4.25 | — | qs to pH 3.75 |
| Sodium Hydroxide | qs to pH 4.25 | qs to pH 4.25 | qs to pH 3.75 | qs to pH 3.75 |
| WFI | qs | qs | qs | qs |

*Equivalent of anhydrous TFA-free form. The actual amount for dispensing should be calculated based upon the water and TFA content according to the COA.

TABLE 6
Bivalirudin Stability (% AUC) over 7 days of Formulations A, F, B, & C

| Purity* (% area) | A (n = 3) | F | B | C |
|---|---|---|---|---|
| Time Zero | 99.4 | 99.5 | 99.4 | 99.4 |
| 12 hours at 60° C. | 92.2 | 93.6 | 72.3 | 77.4 |
| 24 hours at 60° C. | 85.0 | 88.0 | 62.1 | 66.9 |
| 48 hours at 60° C. | 75.7 | 77.7 | 49.9 | 57.1 |
| 7 days at 40° C. | 88.9 | 89.7 | 88.3 | 90.0 |

*Purity (% area) = 100% − total impurity (% area)

TABLE 7
Relative Bivalirudin Stability over 7 days of Formulations
A, F, B, & C as compared to Formulation A

| Composition (mg/mL) | A | F | F/A | B | C | C/B |
|---|---|---|---|---|---|---|
| Pseudo-zero order rate | 100% | 88% | 0.88 | 110% | 95% | 0.86 |
| constant of total impurities | | | | | | |
| at 60° C. | | | | | | |
| Increase in total impurity | 100% | 88% | 0.88 | 113% | 95% | 0.84 |
| over 7 days at 40° C./75% | | | | | | |
| RH | | | | | | |

### Example 3

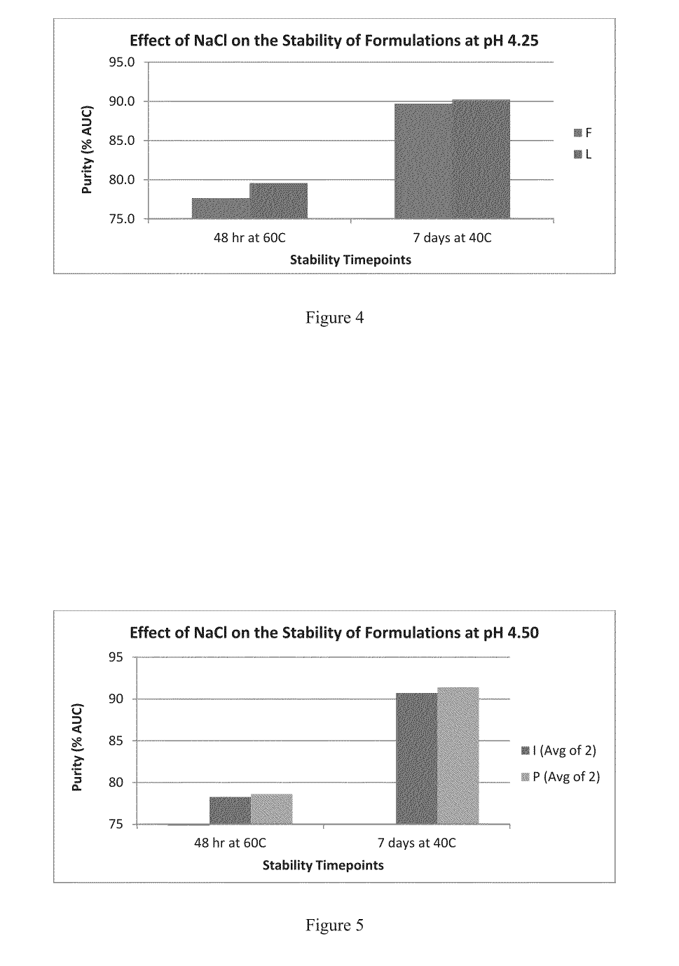

The stability profiles of a bivalirudin solution at two different pH levels in the presence of different concentrations of sodium chloride were measured. The formulations shown in Table 8 were prepared as described above and stored at 60° C. for 48 hours and at 40° C. for 7 days. Purity was measured as described above, and the results are shown in Tables 9 and 10. FIG. 4 depicts the stability profile of bivalirudin as a function of sodium chloride concentration at pH 4.25. FIG. 5 depicts the stability profile of bivalirudin as a function of sodium chloride concentration at pH 4.50.

TABLE 8
Bivalirudin Formulation Composition

| Composition (mg/mL) | K | F | L | I | P |
|---|---|---|---|---|---|
| Bivalirudin* | 5.00 | 5.00 | 5.00 | 5.00 | 5.00 |
| Sodium Chloride | — | 9.00 | 13.00 | 9.00 | 13.00 |
| Hydrochloride Acid | qs to pH 4.25 | qs to pH 4.25 | qs to pH 4.25 | qs to pH 4.50 | qs to pH 4.50 |
| Sodium Hydroxide | qs to pH 4.25 | qs to pH 4.25 | qs to pH 4.25 | qs to pH 4.50 | qs to pH 4.50 |
| WFI | qs | qs | qs | qs | qs |

*Equivalent of anhydrous TFA-free form. The actual amount for dispensing should be calculated based upon the water and TFA content according to the COA.

TABLE 9
Bivalirudin Stability over 7 days for Formulations L, I, and P

| Purity* (% AUC) | L | I (n = 2) | P (n = 2) |
|---|---|---|---|
| Time Zero | 99.5 | 99.4 | 99.2 |
| 48 hr at 60° C. | 79.6 | 78.3 | 78.6 |
| 7 days at 40° C. | 90.2 | 90.7 | 91.4 |

*Purity (% area) = 100% − total impurity (% area)

TABLE 10
Relative Bivalirudin Stability over 7 days of Formulations
L, I, and P as compared to Formulation A

| Composition | | | | | | | | |
|---|---|---|---|---|---|---|---|---|
| (mg/mL) | K | F | F/ K | L | L/K | I | P | P/I |
| Pseudo-zero | 97% | 88% | 0.91 | 91% | 0.94 | 88% | 86% | 0.98 |
| order rate | | | | | | | | |
| constant of | | | | | | | | |
| total impurities | | | | | | | | |
| at 60° C. | | | | | | | | |
| Increase in | 93% | 88% | 0.95 | 89% | 0.96 | 84% | — | — |
| total impurity | | | | | | | | |
| over 7 days at | | | | | | | | |
| 40°C./75% | | | | | | | | |
| RH | | | | | | | | |

### Example 4

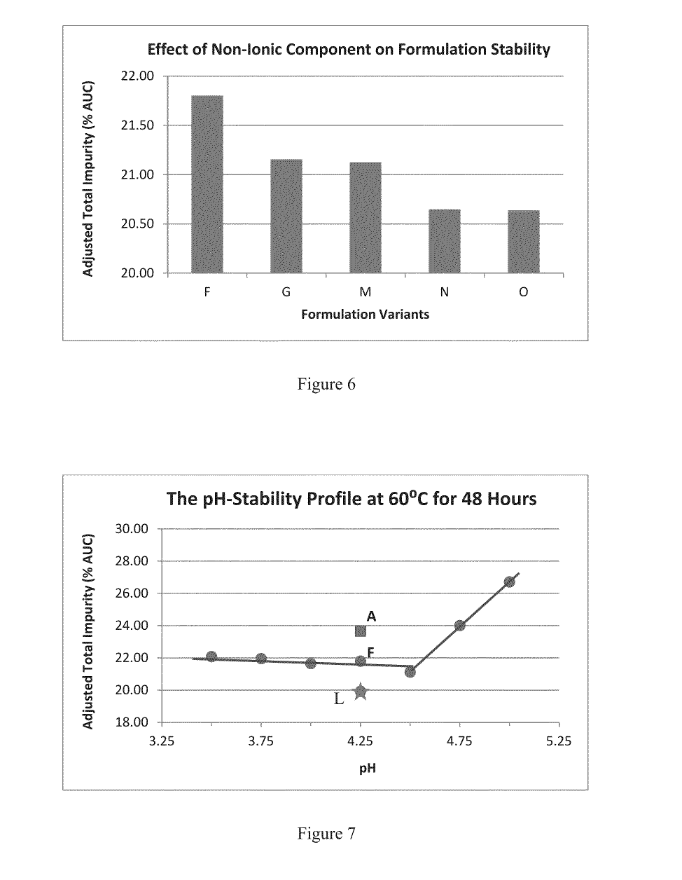

The stability profiles of a bivalirudin solution at pH 4.25 in the presence of sodium chloride, sucrose, dextrose, and/or glycerin and in the absence of a buffer were measured. The formulations shown in Table 11 were prepared as described above and the amounts of sodium chloride, sucrose, dextrose, and glycerin were selected such that the osmolality of the formulations was about 200-600 mOsm/kg. The formulations were stored at 60° C. for 48 hours. Impurities were measured as described above, and the results are shown in Table 12. FIG. 6 depicts the effect of partially or completely replacing sodium chloride with sucrose, dextrose, or glycerin on the stability profile of bivalirudin at pH 4.25.

TABLE 11
Bivalirudin Formulation Composition

| Composition | | | | | |
|---|---|---|---|---|---|
| (mg/mL) | F | G | M | N | O |
| Bivalirudin* | 5 | 5 | 5 | 5 | 5 |
| Sodium | 9 | 6 | — | — | 9 |
| Chloride | | | | | |
| Sucrose | — | — | 160 | — | — |
| Dextrose | — | 50 | — | 75 | — |
| Glycerin | — | — | — | — | 40 |
| Hydrochloric | qs to pH | qs to pH | qs to pH | qs to pH | qs to pH |
| Acid | 4.25 | 4.25 | 4.25 | 4.25 | 4.25 |
| Sodium | qs to pH | qs to pH | qs to pH | qs to pH | qs to pH |
| Hydroxide | 4.25 | 4.25 | 4.25 | 4.25 | 4.25 |
| WFI | Qs | qs | qs | qs | qs |
| Osmolality | | | 588 | 457 | 465 |
| (mOsm/kg) | | | | | |

*equivalent of anhydrous TFA-free form. The actual amount for dispensing should be calculated based upon the water and TFA content according to the COA.

TABLE 12
Adjusted Total Impurities over 7 days for
Formulations F, G, M, N, and O

| Adjusted Total | | | | | |
|---|---|---|---|---|---|
| Impurity (% AUC) | F | G | M | N | O |
| Time Zero | 0.00 | 0.00 | 0.00 | 0.00 | 0.00 |
| 12 hr at 60° C. | 5.86 | 6.10 | 5.41 | 5.73 | 5.87 |
| 24 hr at 60° C. | 11.50 | 11.86 | 11.33 | 11.43 | 11.03 |
| 48 hr at 60° C. | 21.80 | 21.16 | 21.13 | 20.65 | 20.64 |

### Example 5

The stability profiles of a bivalirudin solution at pH ranging from 3.50 to 5.00 in the presence or absence of a buffer and at different concentrations of sodium chloride were measured. Formulations A, F, and L were prepared as described above and the pH of formulation F was varied to range from 3.50 to 5.00. The formulations were stored at 60° C. for 48 hours. Impurities were measured as described above. FIG. 7 depicts the stability profile of bivalirudin as a function of pH, buffer and sodium chloride concentration.

### Example 6

The long term stability of Formulation L was measured. Stoppered and sealed 50 mL clear glass vials containing 50 mL of Formulation L were stored in either an upright or inverted position at refrigeration temperature conditions (2° to 8° C.).

Bivalirudin and bivalirudin-related impurities were measured by HPLC over the course of 12 months. HPLC was performed using gradient of acetonitrile and water with sodium phosphate buffer at a pH of 6.5, a C18 column, a flow rate of 0.3 mL/min, a column temperature of 65° C., and a runtime of 50 minutes. Absorbance at 214 nm was measured, and a PDA detector was used to compare UV spectra. The concentration of bivalirudin was determined using the peak area of bivalirudin as compared to a reference standard of bivalirudin. The assay of the samples is quantified as the % of labeled bivalirudin concentration (% label claim). Bivalirudin-related impurities were also tested by HPLC analysis in the same injection as the assay reported as area percent. The pH of the formulations was also measured regularly over the course of 12 months.

Representative data points are shown in Tables 13A-13F.

TABLE 13A
Upright Sample #1

| | Initial | 3 Months | 6 Months | 12 Months |
|---|---|---|---|---|
| Bivalirudin Assay (% label | 99.1 | 99.8 | 98.1 | 95.9 |
| claim) | | | | |
| Bivalirudin-Related | 1.1 | 2.0 | 2.8 | 4.8 |
| Impurities (area %) | | | | |
| pH | 4.3 | 4.2 | 4.2 | 4.2 |

TABLE 13B
Upright Sample #2

| | Initial | 3 Months | 6 Months | 12 Months |
|---|---|---|---|---|
| Bivalirudin Assay (% label | 99.6 | 99.8 | 98.4 | 96.3 |
| claim) | | | | |
| Bivalirudin-Related | 1.1 | 2.1 | 2.8 | 4.9 |
| Impurities (area %) | | | | |
| pH | 4.3 | 4.1 | 4.2 | 4.2 |

TABLE 13C
Upright Sample #3

| | Initial | 3 Months | 6 Months | 12 Months |
|---|---|---|---|---|
| Bivalirudin Assay (% label | 99.8 | 99.6 | 97.2 | 95.6 |
| claim) | | | | |
| Bivalirudin-Related | 1.9 | 2.4 | 3.2 | 5.3 |
| Impurities (area %) | | | | |
| pH | 4.2 | 4.1 | 4.2 | 4.1 |

TABLE 13D
Inverted Sample #1

| | Initial | 3 Months | 6 Months | 12 Months |
|---|---|---|---|---|
| Bivalirudin Assay (% label | 99.1 | 99.7 | 98.3 | 95.8 |
| claim) | | | | |
| Bivalirudin-Related | 1.1 | 2.0 | 2.8 | 4.7 |
| Impurities (area %) | | | | |
| pH | 4.3 | 4.2 | 4.2 | 4.2 |

TABLE 13E
Inverted Sample #2

| | Initial | 3 Months | 6 Months | 12 Months |
|---|---|---|---|---|
| Bivalirudin Assay (% label | 99.6 | 99.7 | 98.0 | 96.4 |
| claim) | | | | |
| Bivalirudin-Related | 1.1 | 2.0 | 2.8 | 4.8 |
| Impurities (area %) | | | | |
| pH | 4.3 | 4.1 | 4.2 | 4.2 |

TABLE 13F
Inverted Sample #3

| | Initial | 3 Months | 6 Months | 12 Months |
|---|---|---|---|---|
| Bivalirudin Assay (% label | 99.8 | 99.2 | 97.2 | 95.2 |
| claim) | | | | |
| Bivalirudin-Related | 1.9 | 2.4 | 3.1 | 5.3 |
| Impurities (area %) | | | | |
| pH | 4.2 | 4.1 | 4.2 | 4.1 |

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

1.-127. (canceled)

128. An aqueous bivalirudin composition comprising
a) 5 mg/mL bivalirudin, and
b) 8 mg/mL to 14 mg/mL sodium chloride,
wherein the composition is substantially free of buffer and has a pH of about 3.9 to about 4.4.

129. The composition of claim 128 comprising about 8 mg/mL to about 10 mg/mL of sodium chloride.

130. The composition of claim 128 comprising about 12 mg/mL to about 14 mg/mL of sodium chloride.

131. The composition of claim 129 comprising 9 mg/mL of sodium chloride.

132. The composition of claim 130 comprising 13 mg/mL of sodium chloride.

133. The composition of claim 128 having a pH of about 3.9 to about 4.4.

134. The composition of claim 133 having a pH of about 4.2 to about 4.3

135. The composition of claim 134 having a pH of about 4.25

136. The composition of claim 128 wherein the composition is stable.

137. The composition of claim 128 wherein the composition has a shelf-life of at least about 12 months.

138. The composition of claim 128 wherein no more than about 5% of bivalirudin is degraded after storage at 2° C.-8° C. for about 12 months.

139. The composition of claim 128 wherein, the composition retains at least about 96%, or 95%, or 94%, or 93% or 92%, 91%, or 90.0% of bivalirudin after storage at a temperature of about 2° C. to about 8° C. over 3, 6, 9, or 12 months.

140. The composition of claim 128 wherein the composition retains at least about 94% of bivalirudin after storage at a temperature of about 2° C. to about 8° C. over 12 months.

141. The composition of claim 128 wherein the composition retains at least about 95% of bivalirudin after storage at a temperature of about 2° C. to about 8° C. over 12 months.

142. A method of treatment comprising administering a therapeutically effective amount of a composition of claim 128 to a patient in need thereof.

143. Use of a composition of claim 128 as an anticoagulant in a patient in need thereof.
